(19) United States
(12) Patent Application Publication
(10) Pub. No.: US 2023/0172957 A1
(43) Pub. Date: Jun. 8, 2023

(54) USE OF ASTRAGALUS MEDICINAL COMPOSITION FOR PREPARING DRUG FOR ENHANCING CANCER THERAPY

(71) Applicant: PHYTOHEALTH CORPORATION, Taipei (TW)

(72) Inventors: I-Lin LEE, Taipei (TW); Chien-Hsin CHENG, Taipei (TW)

(21) Appl. No.: 17/912,799

(22) PCT Filed: Mar. 17, 2021
(86) PCT No.: PCT/CN2021/081377

§ 371 Date: Sep. 19, 2022

Related U.S. Application Data
(60) Provisional application No. 62/990,650, filed on Mar. 17, 2020.

(51) Int. Cl. A61K 31/7048; A61K 31/7004; A61P 11/00; A61K 31/517; A61K 31/455; A61P 1/00; A61P 15/08; A61K 31/502; A61P 35/00; A61K 36/481

(52) U.S. Cl. CPC A61K 31/7048; A61K 31/7004; A61P 11/00; A61K 31/517; A61K 31/455; A61P 1/00; A61P 15/08; A61K 31/502; A61P 35/00; A61K 36/481

(57) ABSTRACT

A use of pharmaceutical composition for enhancing cancer treatment includes an Astragalus membranaceus extract and a mannose. The pharmaceutical composition can effectively enhance the effectiveness of chemotherapy drugs, thereby reducing cancer cell viability, and for drug resistant cancers. In addition, the pharmaceutical composition can also inhibit the growth of tumor cells.

### CROSS-REFERENCE TO RELATED APPLICATIONS

This application is the U.S. national phase of PCT Application No. PCT/CN2021/081377 filed on Mar. 17, 2021, which claims the benefit of priority under 35 U.S.C. § 119(e) to U.S. Provisional Patent Application No. 62/990,650, filed on Mar. 17, 2020, both of the foregoing patent applications being herein incorporated by reference.

### FIELD OF THE INVENTION

This disclosure relates to a use of a pharmaceutical composition, which comprising a refined astragalus polysaccharides (hereinafter referred to as r-APS) and a mannose, in combination with chemotherapy drugs to enhance the ability of chemotherapy drugs to poison different cancer tumor cells.

### BACKGROUND OF THE INVENTION

The astragalus is Astragalus mongholicus Bunge or Astragalus mongholicus, to be specific, the astragalus refers to the dry root of Astragalus membranaceus Bge. var. mongholicus (Bge.) Hsiao or A. membranaceus (Fisch.) Bge. According to the record of traditional pharmacy, the astragalus is used for treating chronic nephritis, proteinuria, myositis, hypertension, coronary heart disease, cerebral infarction, peptic ulcer (duodenal and gastric ulcers), the kidney disease and diabetes. Additionally, a few kinds of r-APS can be used for treating tumors. Some studies show that the tumor treatment mechanisms include adjusting immune response (Lau, B. H. S., Ruckle, H. C. Botolazzo, T.) and “using Chinese herbal medicine to inhibit the growth of mouse's renal-cell carcinoma” of Lui, P. D.

The composition of astragalus has been analyzed in recent years, including monosaccharide, polysaccharide, flavone, amino acid and trace element. The content of polysaccharide is the highest and the treatment effect of polysaccharide on diseases has been proved, especially on the cancer related diseases, e.g. tumors (U.S. Pat. No. 5,268,467; “recent development of antineoplastic agent of science of Chinese herbal medicine” of Tang, W. Hemm, I. and Bertram, B.), Part II, high molecular weight compounds.

For rapid growth, the malignant tumor consumes more glucose than normal healthy tissues, it is hard to control internal glucose content only by diet. The Cancer Research UK Beatson Institute indicates that “the tumor needs a lot of glucose for growth, so the cancer development can be slowed down by restricting the available glucose for tumor. The question is that normal tissues need glucose, too. Therefore, we cannot remove glucose thoroughly. It was found in research that a certain dose of mannose could block a considerable amount of glucose, so as to slow down the mouse's tumor growth, and the normal tissues would not be affected. That was an early study. If a perfect balance can be found in the future, the mannose can enhance the chemotherapeutic effect on cancer patients without damaging their overall health”. The mannose is a hexose, existing in human blood in minute quantities, it is known that it is supplied not by ingesting food, but by glucose metabolism.

Although the techniques of targeted drugs and precision medicine are increasingly mature, the drug resistance is still a problem in cancer treatment. If a targeted chemotherapy drug is used in high dose or for a long time, the cancer cells often acquire drug resistance, and survive and keep growing inside the host, leading to chemotherapy drug ineffective cancer treatment eventually. The chemotherapy drug is effective at the initial stage of treatment, at the initial stage of administration, the tumor growth can be inhibited effectively, but after a period of time, the tumor grows again. As the treatment goes on, the drug begins to fail, this phenomenon is medically known as drug resistance. The drug resistance not only makes the cancer recur, but also puts the patient in such a desperate situation that none of drugs is workable, reducing the side effects of chemotherapy drugs and the drug resistance of cancers is an important topic of cancer treatment.

Accordingly, it is very important to develop an effective, safe and reversing drug resistant pharmaceutical composition.

### SUMMARY OF THE INVENTION

In present invention, it is found that an astragalus pharmaceutical composition, which comprising a refined astragalus polysaccharides (hereinafter referred to as r-APS) and a mannose, for the patient receiving chemotherapy, the amount of chemotherapy drug equivalent to the original dose is used in comparison to using the r-APS alone or using the mannose alone, but the effect of chemotherapy drug on reducing the viability of cancer cells can be enhanced, so the therapeutic effect of chemotherapy drug can be enhanced effectively.

The astragalus pharmaceutical composition, which comprising a r-APS and a mannose of the present invention, for the patient with chemotherapy drug resistance, only needs amount of chemotherapy drug equivalent to the original dose, but the effect of chemotherapy drug on reducing the viability of cancer cells can be increased by at least 16%, so even if the patient has been resistant to drug, the therapeutic effect of chemotherapy drug can be enhanced effectively.

In view of the above-mentioned problem, the present invention provides a method for enhancing the effect of a chemotherapy drug for a cancer in a subject on inhibiting the viability of a cancer cell, comprising administering to the subject an astragalus pharmaceutical composition, wherein the astragalus pharmaceutical composition comprises a r-APS and a mannose.

In some embodiments, the chemotherapy drug for the cancer is a targeted chemotherapy drug, the targeted chemotherapy drug is a targeted chemotherapy drug for the corresponding cancer type.

In some embodiments, the cancer comprises colorectal cancer, ovarian cancer, or lung cancer.

In some embodiments, the targeted chemotherapy drug for corresponding cancer type comprises Regorafenib, Olaparib or Tarceva.

In some embodiments, an effective dose ratio by weight of the r-APS to the mannose in the astragalus pharmaceutical composition is 1.5˜4.5:5.5˜8.5, and a concentration range of the combination of the r-APS and the mannose is 0˜10 mg/mL.

In some embodiments, the astragalus pharmaceutical composition can be tablet, pill, granule, powder, capsule, or liquid.

Further, the present invention also provides a method for enhancing the effect of a chemotherapy drug for a cancer in a subject on treating a drug resistant cancer on inhibiting the viability of a cancer cell, comprising administering to the subject an astragalus pharmaceutical composition, wherein the astragalus pharmaceutical composition comprises a r-APS and a mannose.

In one embodiment, the cancer is a solid tumor cancer.

In one embodiment, the solid tumor cancer comprises colorectal cancer, ovarian cancer, or lung cancer.

Further, the present invention also provides a method for enhancing the effect of a chemotherapy drug for cancer in a subject on inhibiting the proliferation of a tumor cell, comprising administering to the subject an astragalus pharmaceutical composition, wherein the astragalus pharmaceutical composition comprises a r-APS and a mannose.

In one embodiment, the tumor cell is a drug resistant tumor cell.

In one embodiment, the tumor cell comprises colorectal cancer, ovarian cancer, or lung cancer.

It is to be understood that both the foregoing general description and the following detailed description are exemplary and explanatory only and are not restrictive of the invention.

### BRIEF DESCRIPTION OF THE DRAWINGS

FIG. 1A shows that the results of the r-APS on drug resistant cell line of lung cancer (HCC827GR cells).

FIG. 1B shows that the results of the astragalus pharmaceutical composition comprising the r-APS and the mannose on drug resistant cell line of lung cancer (HCC827GR cells).

FIG. 1C shows that the results of Iressa as chemotherapy drug for lung cancer on drug resistant cell line of lung cancer (HCC827GR cells).

FIG. 2A shows that the results of the r-APS on drug resistant cell line of lung cancer (H1975 cells).

FIG. 2B shows that the results of the astragalus pharmaceutical composition comprising the r-APS and the mannose on drug resistant cell line of lung cancer (H1975 cells).

FIG. 2C shows that the results of Tarceva as chemotherapy drug for lung cancer on drug resistant cell line of lung cancer (H1975 cells).

FIG. 3A shows that the results of the r-APS in combination with Tarceva as chemotherapy drug for lung cancer on drug resistant cells of lung cancer (H1975 cells).

FIG. 3B shows that the results of the astragalus pharmaceutical composition comprising the r-APS and the mannose in combination with Tarceva as chemotherapy drug for lung cancer on drug resistant cell line of lung cancer (H1975 cells).

FIG. 4A shows that the select results of the 60˜70% chemotherapeutic inhibition cultured with cell line of colorectal cancer (HCT116 cells).

FIG. 4B shows that the results of cell line of colorectal cancer (HCT116 cells) cultured with the r-APS for 48 hours.

FIG. 4C shows that the results of cell line of colorectal cancer (HCT116 cells) cultured with the astragalus pharmaceutical composition comprising the r-APS and the mannose for 48 hours.

FIG. 4D shows that the results of cell line of colorectal cancer (HCT116 cells) cultured with the r-APS in combination with Regorafenib as chemotherapy drug for colorectal cancer for 48 hours.

FIG. 4E shows that the results of cell line of colorectal cancer (HCT116 cells) cultured with the astragalus pharmaceutical composition comprising the r-APS and the mannose in combination with Regorafenib as chemotherapy drug for colorectal cancer for 48 hours.

FIG. 5A shows that the select results of 60˜70% chemotherapeutic inhibition cultured with ovarian cancer cell line (OVCAR3 cells).

FIG. 5B shows that the results of ovarian cancer cell line (OVCAR cells) cultured with the r-APS for 48 hours.

FIG. 5C shows that the results of ovarian cancer cell line (OVCAR cells) cultured with the astragalus pharmaceutical composition comprising the r-APS and the mannose for 48 hours.

FIG. 5D shows that the results of ovarian cancer cell line (OVCAR cells) cultured with the r-APS in combination with Olaparib as chemotherapy drug for colorectal cancer for 48 hours.

FIG. 5E shows that the result of the astragalus pharmaceutical composition comprising the r-APS and the mannose in combination with Olaparib as chemotherapy drug for colorectal cancer cultured with ovarian cancer cell line (OVCAR cells) for 48 hours.

### DETAILED DESCRIPTION OF THE INVENTION

The “r-APS” in the present invention refers to refined astragalus polysaccharides.

The enhancement of chemotherapy drug in the present invention is enhanced at least 16% of therapeutic effect.

The chemotherapy drug in the present invention comprises targeted chemotherapy drug, and the targeted chemotherapy drug is a targeted chemotherapy drug for corresponding cancer type.

The present invention provides a method for enhancing the treatment of a chemotherapy drug for a cancer in a subject for inhibiting the viability of a cancer cell, comprising administering to the subject an astragalus pharmaceutical composition, wherein the astragalus pharmaceutical composition comprises a r-APS and a mannose, wherein the ratio by weight of the r-APS to the mannose in the astragalus pharmaceutical composition is 1.5˜4.5:5.5˜8.5. The effective concentration range of the r-APS of the astragalus pharmaceutical composition in present invention is 0˜10 mg/mL, the effective concentration range of the mannose is 0˜10 mg/mL. Preferably, the effective concentration of the r-APS is 3 mg/mL, the effective concentration of the mannose is 7 mg/mL.

Further, the present invention also provides a method for enhancing or maintaining the treatment of a chemotherapy drug for a cancer in a subject for treating a drug resistant cancer on inhibiting the viability of a cancer cell, comprising administering to the subject an astragalus pharmaceutical composition, wherein the astragalus pharmaceutical composition comprises a r-APS and a mannose, wherein the ratio by weight of the r-APS to the mannose in the astragalus pharmaceutical composition is 1.5˜4.5:5.5˜8.5. The effective concentration range of r-APS in the astragalus pharmaceutical composition of the present invention is 0˜10 mg/mL, the effective concentration range of mannose is 0˜10 mg/mL. Preferably, the effective concentration of r-APS is 3 mg/mL, the effective concentration of mannose is 7 mg/mL.

Further, the present invention also provides a method for enhancing the treatment of a chemotherapy drug for a cancer in a subject for inhibiting the proliferation of a tumor cell, comprising administering to the subject an astragalus pharmaceutical composition, wherein the astragalus pharmaceutical composition comprises a r-APS and a mannose, wherein the weight ratio of the r-APS to the mannose in the astragalus pharmaceutical composition is 1.5˜4.5:5.5˜8.5. The effective concentration range of the r-APS in the astragalus pharmaceutical composition of the present invention is 0˜10 mg/mL, the effective concentration range of the mannose is 0˜10 mg/mL. Preferably, the effective concentration of the r-APS is 3 mg/mL, the effective concentration of the mannose is 7 mg/mL.

In some embodiments, the astragalus pharmaceutical composition is administered by injection, infusion, intravenous, or oral.

In some preferred embodiments of the present invention, the method for manufacturing the r-APS in present invention comprising the steps of:

(a) Put the astragalus chips in a container, add a pure water to form a solution A;
(b) Heat the solution A formed in step (a) at a time in 2-4 hours, the temperature is 80-100° C.;
(c) Collect and concentrate the solution of step (b) to obtain an extraction liquid A;
(d) Put the extraction liquid A of step (c) in a container B, add 30˜50% ethanol, stir and precipitate, collect and concentrate the supernatant to obtain a concentrated solution A;
(e) Add 75%˜95% ethanol in the concentrated solution A obtained in step (d), stir and precipitate, collect the precipitate A; and
(f) Dehydrate and filter the precipitate A of step (e), dissolve and precipitate with ethanol, centrifuge the precipitate to obtain product A, the product A contains r-APS.

Additional specific embodiments of the present invention include, but are not limited to the following:

### Example 1

Influence of the r-APS and the Mannose on Drug Resistant Cells of Lung Cancer

This example discusses the influence of the r-APS and the mannose on the drug resistant cells of lung cancer, there are three test groups, respectively treated as follows:

(1) Test group 1: the different concentrations of the r-APS are 0.25 mg/ml, 0.5 mg/ml, 1 mg/ml, 2 mg/ml and 4 mg/ml.
(2) Test group 2: the different concentrations of the astragalus pharmaceutical composition comprising the r-APS and the mannose (hereinafter referred to as r-APS+mannose) (the ratio by weight of the r-APS:the mannose is 30%:70%) are 0.625 mg/ml, 1.25 mg/ml, 2.5 mg/ml, 5 mg/ml and 10 mg/ml.
(3) Test group 3: Iressa (applicable to the HCC827GR cell line) or Tarceva (applicable to the H1975 cell line).

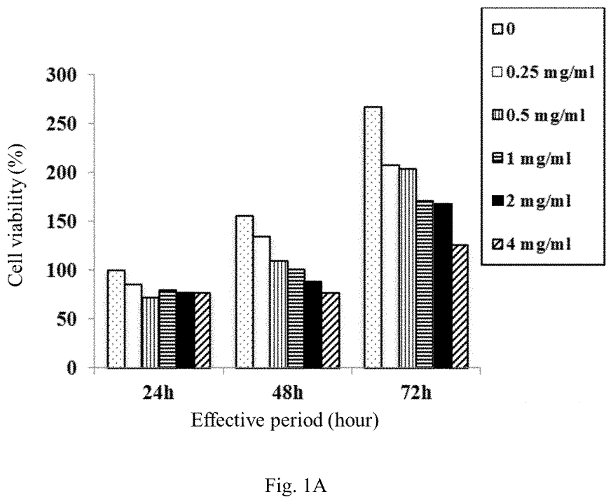

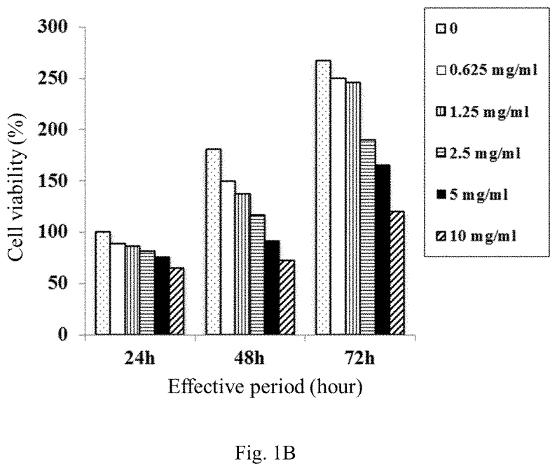

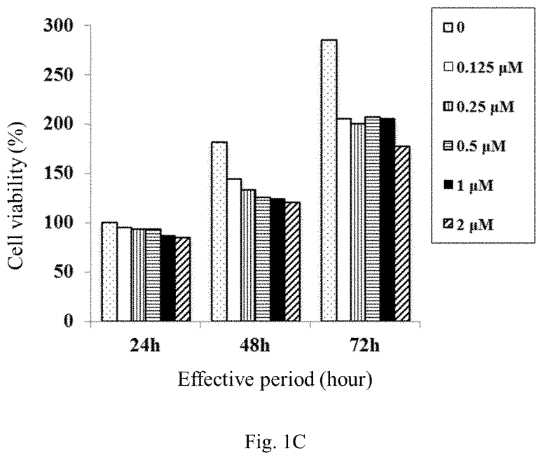

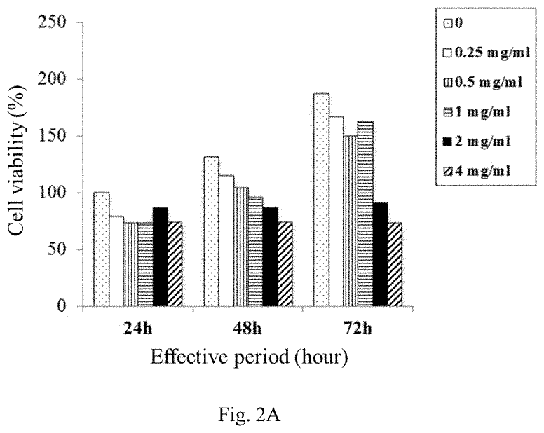

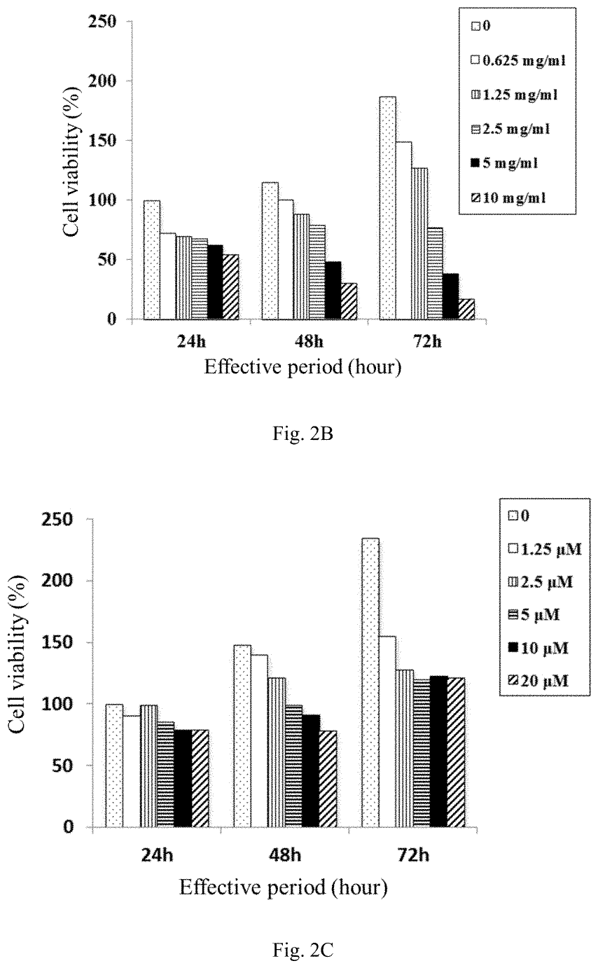

The test group 1, test group 2 and test group 3 are added to the HCC827GR or the H1975 lung cancer cell line respectively, each group incubates for 24 hours, 48 hours and 72 hours respectively, and then the cell viability of the HCC827GR or the H1975 lung cancer cell line is tested. The results are shown in FIG. 1A˜1C and FIG. 2A˜2C.

The HCC827GR cell line is resistant to the chemotherapy drug Iressa for treating lung cancer, the result shows that the test group 2 (the r-APS) and test group 3 (the astragalus pharmaceutical composition comprising the r-APS and the mannose (r-APS+mannose)) have better anticancer effect to lung cancer (as shown in FIG. 1A˜FIG. 1C).

The H1975 cell line is a cell line resistant to the drug Tarceva for treating lung cancer, the result shows that the test group 3 (the astragalus pharmaceutical composition comprising the r-APS and the mannose (r-APS+mannose)) has the best anticancer effect (as shown in FIG. 2A˜FIG. 2C).

### Example 2

Effect of the Astragalus Pharmaceutical Composition Comprising the r-APS and the Mannose (r-APS+Mannose) in Combination with Anticancer Drug on the Drug Resistant Cells of Lung Cancer

This example discusses the influence of the astragalus pharmaceutical composition comprising the r-APS and the mannose (r-APS+mannose) on the drug resistant cells of lung cancer, there are three test groups, respectively treated as follows:

(1) Test group 1: the different concentrations of the r-APS are 0.25 mg/ml, 0.5 mg/ml, 1 mg/ml, 2 mg/ml and 4 mg/ml.
(2) Test group 2: the different concentrations of the astragalus pharmaceutical composition comprising the r-APS and the mannose (r-APS+mannose) (the ratio by weight of the r-APS:the mannose is 30%:70%) are 0.625 mg/ml, 1.25 mg/ml, 2.5 mg/ml, 5 mg/ml and 10 mg/ml.

Each test group is used with Tarceva (concentrations are 1.25 μM, 2.5 μM and 5 μM) and added to H1975 lung cancer cell line respectively, each group incubates for 24 hours, 48 hours and 72 hours respectively, the cell viability of H1975 lung cancer cell line is measured.

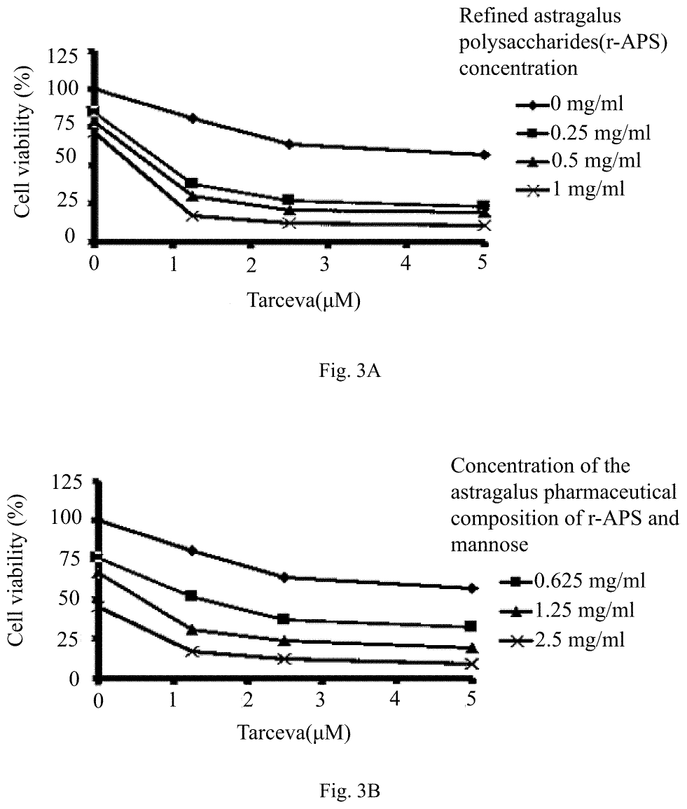

The H1975 cell line is resistant to chemotherapy drug Tarceva, the result shows that the test group 1 (the r-APS) and test group 2 (the astragalus pharmaceutical composition comprising the r-APS and the mannose (r-APS+mannose)) in combination with chemotherapy drug Tarceva have the best anticancer effect (as shown in FIGS. 3A-3B), meaning the test group 1 (the r-APS) and test group 2 (the astragalus pharmaceutical composition comprising the r-APS and the mannose (r-APS+mannose)) in combination with chemotherapy drug Tarceva have the best therapeutic effect on reversing the drug resistance to anticancer drugs of lung cancer.

### Example 3

Influence of the Astragalus Pharmaceutical Composition of the Invention on Colorectal Cancer

Experimental Method:

(1) First of all, poisonous dose test (the chemotherapy drug dose for inhibiting the cell growth and leaving 60˜70% of growth capability is selected for subsequent test)
(2) The cells are inoculated in 96-well plate and cultured for 24 hours.
(3) Test drugs at different concentrations are added in, the test concentrations of the r-APS are 0, 2 and 4 mg/ml, the test concentrations of the astragalus pharmaceutical composition comprising the r-APS and the mannose (r-APS+mannose) are 0, 5 and 10 mg/ml.
(4) The dose with a poisoning effect on 35˜40% of individual cells is selected as chemotherapy drug dose.
(5) The cell viability is tested in 48 and 72 hours respectively after addition.
(6) Experiment on chemotherapeutic synergism of the r-APS and the astragalus pharmaceutical composition comprising the r-APS and the mannose (r-APS+mannose):
(7) The cells are inoculated in 96-well plate and cultured for 24 hours.
(8) The r-APS or the astragalus pharmaceutical composition comprising the r-APS and the mannose (r-APS+mannose) in combination with chemotherapy drug are cultured for 48 hours, the doses of the r-APS and the astragalus pharmaceutical composition comprising the r-APS and the mannose (r-APS+mannose) are selected four poisonous doses for test, and then the cell viability is measured by MTS in 48 hours.

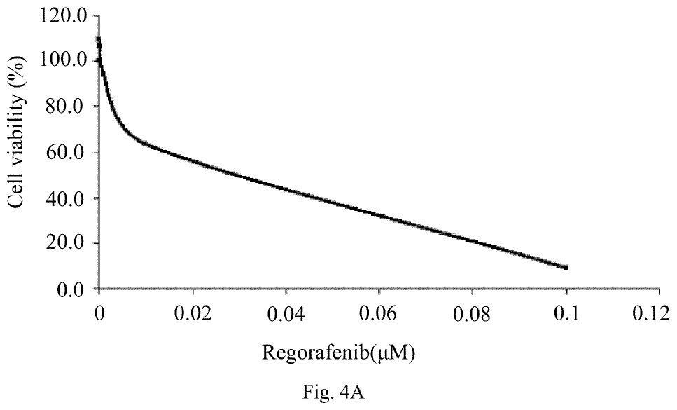

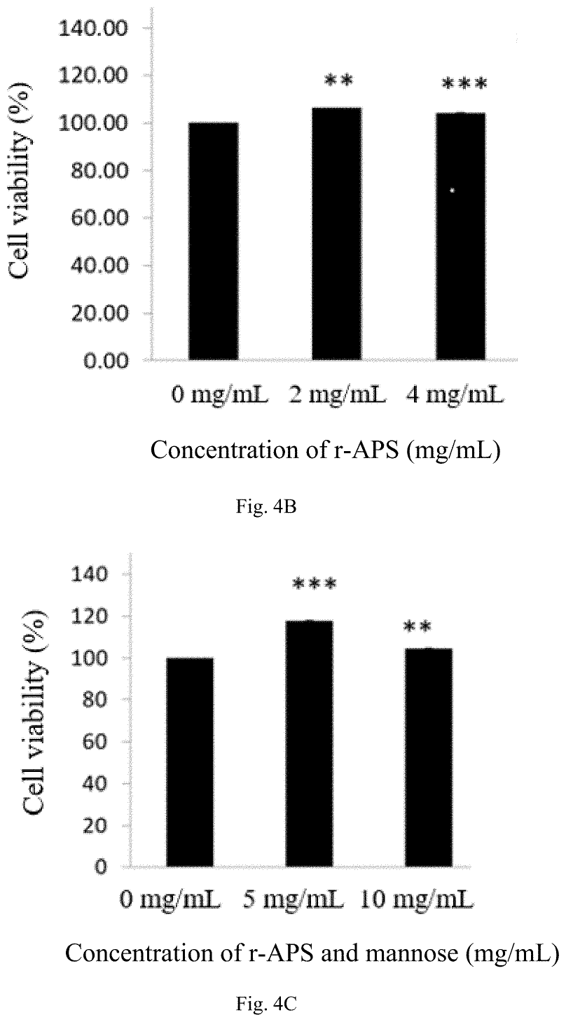

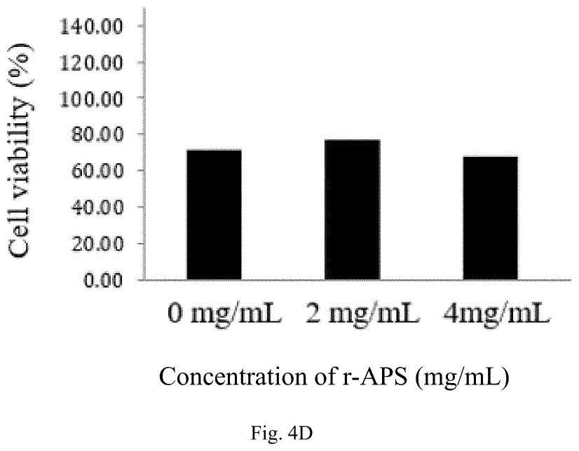

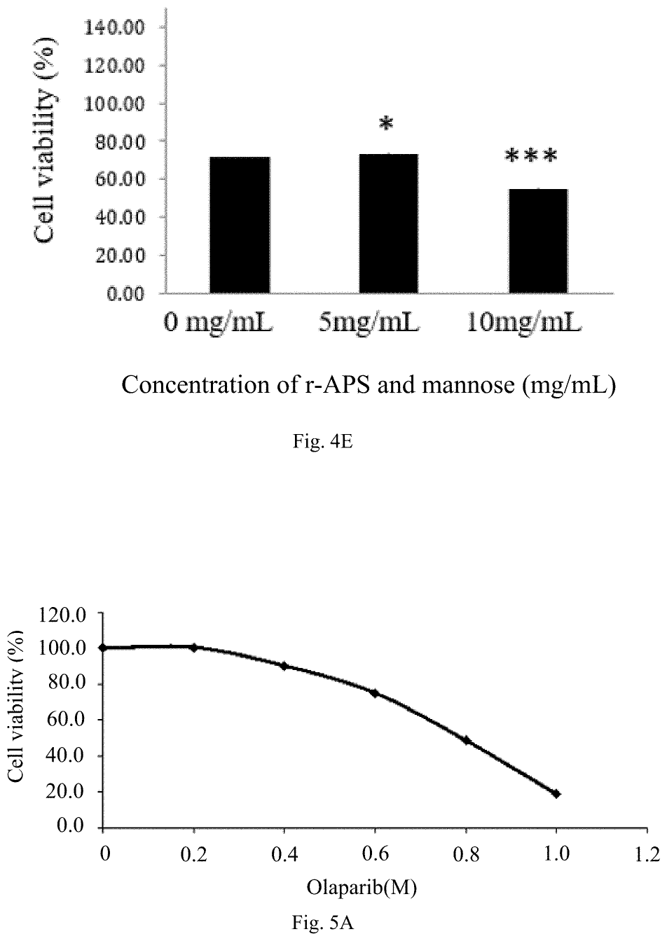

Poisonous dose test result: as shown in Table 1, when the dose of chemotherapy drug Regorafenib is 0.01 mM, the cell viability of the cancer cells can be poisoned to 60%, the condition after the cancer patient takes the chemotherapy drug is simulated, the experimental result of chemotherapeutic synergism of the r-APS and the astragalus pharmaceutical composition comprising the r-APS and the mannose (r-APS+mannose) is shown in FIG. 4A.

TABLE 1
Cell viability (%) of cancer cells of colorectal cancer poisoned
by chemotherapy drug Regorafenib at different concentrations

| | Concentration | | | | | |
|---|---|---|---|---|---|---|
| | 0 | 0.00001 | 0.0001 | 0.001 | 0.01 | 0.1 |
| Repeat | (mM) | (mM) | (mM) | (mM) | (mM) | (mM) |
| Repeat 1 | 1.054 | 1.108 | 1.102 | 1.095 | 0.646 | 0.094 |
| Repeat 2 | 0.973 | 1.1 | 1.137 | 0.849 | 0.634 | 0.095 |
| Repeat 3 | 0.991 | 1.1 | 0.985 | 0.906 | 0.637 | 0.089 |
| Mean value | 1.006 | 1.103 | 1.075 | 0.950 | 0.639 | 0.093 |
| (MEAN) | | | | | | |
| Standard | 0.04 | 0.00 | 0.08 | 0.13 | 0.01 | 0.00 |
| deviation | | | | | | |
| (SD) | | | | | | |
| Cell viability | 100.0 | 109.6 | 106.8 | 94.4 | 63.5 | 9.2 |
| (%) | | | | | | |

As shown below in Table 2, the cancer cells of colorectal cancer (HCT116 cells) are added in the r-APS and cultured for 48 hours, the result shows that the growth of cancer cells is promoted slightly when the r-APS is used alone, meaning using the r-APS alone has no significant poisoning effect (as shown in FIG. 4B).

TABLE 2
Cell viability (%) of cancer cells of colorectal
cancer (HCT116 cells) culture for 48 hours after
adding the r-APS at different concentrations.

| | Concentration | | |
|---|---|---|---|
| | 0 | 2 | 4 |
| Repeat | (mg/ml) | (mg/ml) | (mg/ml) |
| Repeat 1 | 1.282 | 1.351 | 1.341 |
| Repeat 2 | 1.290 | 1.381 | 1.342 |
| Repeat 3 | 1.294 | 1.374 | 1.347 |
| Mean value (MEAN) | 1.289 | 1.369 | 1.343 |
| Standard deviation (SD) | 0.01 | 0.02 | 0.00 |
| Cell viability (%) | 100.00 | 106.21 | 104.24 |

As shown below in Table 3, the HCT116 has been treated with the astragalus pharmaceutical composition comprising the r-APS and the mannose (r-APS+mannose) for 48 hours, the result shows that the astragalus pharmaceutical composition comprising the r-APS and the mannose (r-APS+mannose) promotes the growth of cancer cells on the contrary, meaning the astragalus pharmaceutical composition comprising the r-APS and the mannose (r-APS+mannose) has no significant poisoning effect (as shown in FIG. 4C).

TABLE 3
Cell viability (%) of cancer cells of colorectal cancer (HCT116
cells) culture for 48 hours after adding the astragalus pharmaceutical
composition comprising the r-APS and the mannose (r-APS +
mannose) at different concentrations.

| | Concentration | | |
|---|---|---|---|
| | 0 | 5 | 10 |
| Repeat | (mg/ml) | (mg/ml) | (mg/ml) |
| Repeat 1 | 1.282 | 1.524 | 1.328 |
| Repeat 2 | 1.290 | 1.524 | 1.361 |
| Repeat 3 | 1.294 | 1.506 | 1.355 |
| Mean value (MEAN) | 1.289 | 1.518 | 1.348 |
| Standard deviation (SD) | 0.01 | 0.010 | 0.018 |
| Cell viability (%) | 100 | 117.80 | 104.60 |

As shown below in Table 4, the cancer cells of colorectal cancer (HCT116 cells) have been cultured for 48 hours after adding chemotherapy drug Regorafenib (0.01 mM) and the r-APS, the result shows that the growth of cancer cells is promoted when the effective concentration of the r-APS is 2 mg/mL, and the growth of cancer cells is inhibited slightly when the concentration is 4 mg/mL, meaning the r-APS in combination with chemotherapy drug has no significant poisoning effect (as shown in FIG. 4D).

TABLE 4
Cell viability (%) of cancer cells of colorectal cancer (HCT116
cells) culture for 48 hours after adding chemotherapy drug Regorafenib
(0.01 mM) and the r-APS at different concentrations.

| | Concentration | | |
|---|---|---|---|
| | 0 | 2 | 4 |
| Repeat | (mg/ml) | (mg/ml) | (mg/ml) |
| Repeat 1 | 0.634 | 0.625 | 0.606 |
| Repeat 2 | 0.646 | 0.710 | 0.614 |
| Repeat 3 | 0.643 | 0.735 | 0.604 |
| Mean value (MEAN) | 0.641 | 0.690 | 0.608 |
| Standard deviation (SD) | 0.01 | 0.06 | 0.01 |
| Cell viability (%) | 71.4 | 76.9 | 67.8 |

As shown below in Table 5, the HCT116 has been cultured for 48 hours after adding chemotherapy drug Regorafenib (0.01 mM) and the astragalus pharmaceutical composition of the r-APS and the mannose (r-APS+mannose), the result shows that when the effective dose of the astragalus pharmaceutical composition comprising the r-APS and the mannose (r-APS+mannose) is 10 mg/mL, the combination of chemotherapy drug can effectively reduce the viability of cancer cells, meaning the astragalus pharmaceutical composition comprising the r-APS and the mannose (r-APS+mannose) in combination with chemotherapy drug has a significant poisoning effect (as shown in FIG. 4E).

TABLE 5
Cell viability (%) of cancer cells of colorectal cancer (HCT116
cells) culture for 48 hours after adding chemotherapy drug Regorafenib
(0.01 mM) and the astragalus pharmaceutical composition comprising
the r-APS and the mannose (r-APS + mannose).

| | Concentration | | |
|---|---|---|---|
| | 0 | 5 | 10 |
| Repeat | (mg/ml) | (mg/ml) | (mg/ml) |
| Repeat 1 | 0.634 | 0.661 | 0.485 |
| Repeat 2 | 0.646 | 0.670 | 0.495 |
| Repeat 3 | 0.643 | 0.653 | 0.497 |
| Mean value (MEAN) | 0.641 | 0.661 | 0.492 |
| Standard deviation (SD) | 0.006 | 0.01 | 0.01 |
| Cell viability (%) | 71.43 | 73.70 | 54.90 |

The experimental results show that the astragalus pharmaceutical composition comprising the r-APS and the mannose (r-APS+mannose) in combination with chemotherapy drug of the present invention can further reduce the viability of cancer cells of colorectal cancer by 16% compared with only using drug, so the therapeutic effect of chemotherapy drug can be enhanced effectively.

### Example 4

Influence of the Astragalus Pharmaceutical Composition of the Present Invention on Ovarian Cancer

Experimental Method:

(1) Poisonous dose test (the chemotherapy drug dose for inhibiting the cell growth and leaving 60˜70% of growth capability is selected for subsequent test).
(2) The cells are inoculated in 96-well plate and cultured for 24 hours.
(3) Test drugs at different concentrations are added in, the test concentrations of the r-APS are 0, 2 and 4 mg/ml, the test concentrations of the astragalus pharmaceutical composition comprising the r-APS and the mannose (r-APS+mannose) are 0, 5 and 10 mg/ml.
(4) The dose with a poisoning effect on 35-40% of individual cells is selected as chemotherapy drug dose.
(5) The cell viability is tested in 48 and 72 hours respectively after addition.
(6) Experiment on chemotherapeutic synergism of the r-APS and the astragalus pharmaceutical composition comprising the r-APS and the mannose (r-APS+mannose).
(7) The cells are inoculated in 96-well plate and cultured for 24 hours.
(8) The r-APS or the astragalus pharmaceutical composition comprising the r-APS and the mannose (r-APS+mannose) in combination with chemotherapy drug are cultured for 48 hours, the dose of the r-APS and the astragalus pharmaceutical composition comprising the r-APS and the mannose (r-APS+mannose) is three poisonous doses for test, the cell viability is measured by MTS in 48 hours.

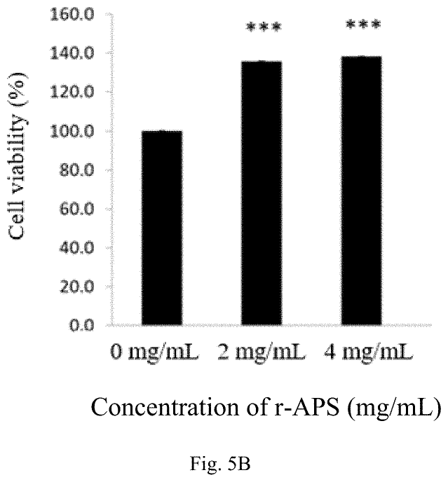

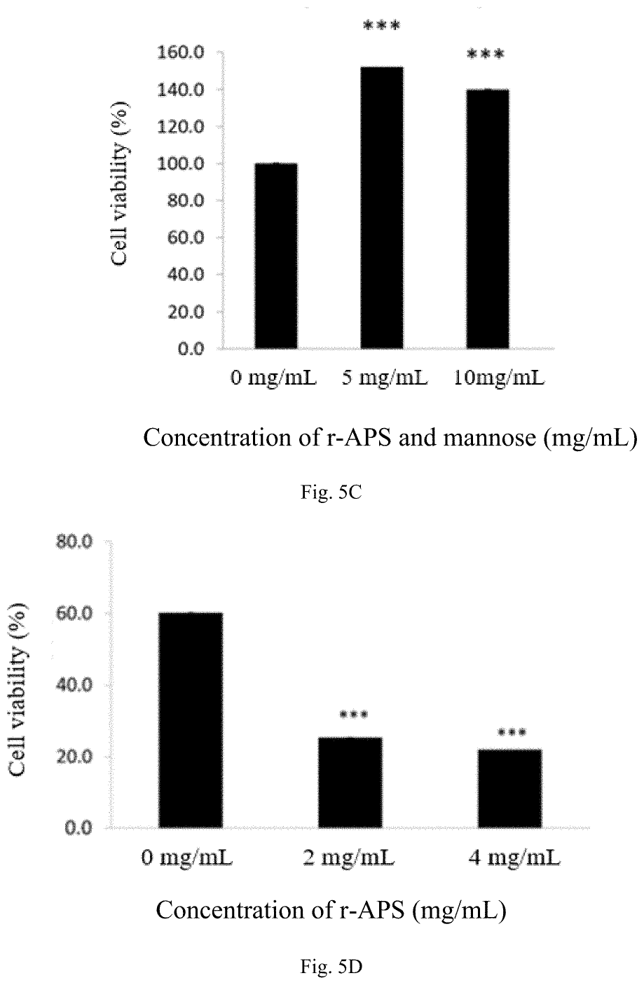

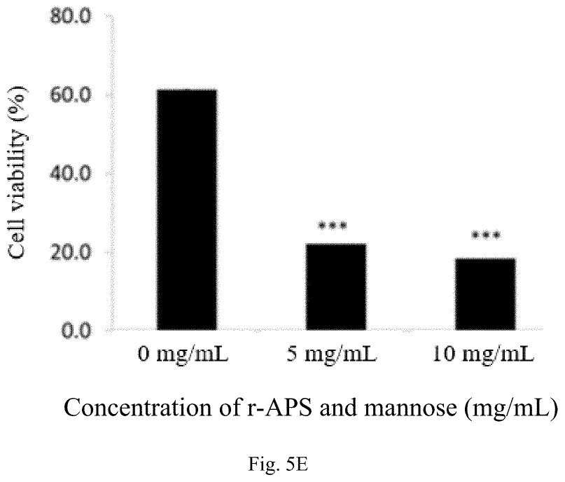

The poisonous dose test result is shown below in Table 6, when the dose of chemotherapy drug Olaparib is 0.7M, the cell viability of cancer cells can be poisoned to 60%, the condition after the cancer patient takes the chemotherapy drug is simulated, the experimental result of chemotherapeutic synergism of the r-APS and the astragalus pharmaceutical composition comprising the r-APS and the mannose (r-APS+mannose) is shown in FIG. 5A.

TABLE 6
Cell viability (%) of ovarian cancer cell line (OVCAR cells) poisoned
by chemotherapy drug Olaparib at different concentrations

| | Concentration | | | | | |
|---|---|---|---|---|---|---|
| Repeat | 0M | 0.2M | 0.4M | 0.6M | 0.8M | 1M |
| Repeat 1 | 0.984 | 0.984 | 0.895 | 0.752 | 0.531 | 0.214 |
| Repeat 2 | 0.982 | 0.991 | 0.871 | 0.738 | 0.413 | 0.152 |
| Repeat 3 | 0.969 | 0.976 | 0.885 | 0.715 | 0.486 | 0.184 |
| Mean value | 0.978 | 0.984 | 0.884 | 0.735 | 0.477 | 0.183 |
| (MEAN) | | | | | | |
| Standard | 0.008 | 0.008 | 0.012 | 0.019 | 0.060 | 0.031 |
| deviation | | | | | | |
| (SD) | | | | | | |
| Cell viability | 100.00 | 100.55 | 90.32 | 75.13 | 48.72 | 18.74 |
| (%) | | | | | | |

The experimental result of chemotherapeutic synergism of the r-APS and the astragalus pharmaceutical composition comprising the r-APS and the mannose (r-APS+mannose) is shown in Table 7, the ovarian cancer cell line (OVCAR cells) is cultured with the r-APS for 48 hours, the result shows that using the r-APS alone promotes the growth of cancer cells on the contrary, meaning using the r-APS alone has no significant poisoning effect (as shown in FIG. 5B)

TABLE 7
Cell viability (%) of ovarian cancer cell line (OVCAR cells) culture
for 48 hours after adding the r-APS at different concentrations.

| | Concentration | | |
|---|---|---|---|
| | 0 | 2 | 4 |
| Repeat | (mg/ml) | (mg/ml) | (mg/ml) |
| Repeat 1 | 0.874 | 1.165 | 1.198 |
| Repeat 2 | 0.873 | 1.215 | 1.237 |
| Repeat 3 | 0.877 | 1.183 | 1.193 |
| Mean value (MEAN) | 0.875 | 1.188 | 1.209 |
| Standard deviation (SD) | 0.002 | 0.025 | 0.024 |
| Cell viability (%) | 100.0 | 135.8 | 138.3 |

As shown below in Table 8, the ovarian cancer cell line (OVCAR cells) has been cultured for 48 hours after adding the astragalus pharmaceutical composition comprising the r-APS and the mannose (r-APS+mannose), the result shows that the astragalus pharmaceutical composition comprising the r-APS and the mannose (r-APS+mannose) promotes the growth of cancer cells, meaning using the astragalus pharmaceutical composition comprising the r-APS and the mannose (r-APS+mannose) alone has no significant poisoning effect (as shown in FIG. 5C).

TABLE 8
Cell viability (%) of ovarian cancer cell line (OVCAR
cells) culture for 48 hours after adding the astragalus
pharmaceutical composition comprising the r-APS and the
mannose (r-APS + mannose) at different concentrations

| | Concentration | | |
|---|---|---|---|
| | 0 | 5 | 10 |
| Repeat | (mg/ml) | (mg/ml) | (mg/ml) |
| Repeat 1 | 0.874 | 1.318 | 1.211 |
| Repeat 2 | 0.877 | 1.351 | 1.251 |
| Repeat 3 | 0.877 | 1.323 | 1.213 |
| Mean value (MEAN) | 0.876 | 1.331 | 1.225 |
| Standard deviation (SD) | 0.002 | 0.018 | 0.023 |
| Cell viability (%) | 100.2 | 152.1 | 140.1 |

As shown in Table 9, the ovarian cancer cell line (OVCAR cells) has been cultured with Olaparib (0.7 M) and the r-APS for 48 hours, the result shows that the r-APS in combination with chemotherapy drug Olaparib can effectively inhibit the viability of ovarian cancer cell line (OVCAR cells), so the r-APS in combination with chemotherapy drug has a significant poisoning effect (as shown in FIG. 5D).

TABLE 9
Cell viability (%) of ovarian cancer cell line (OVCAR cells) culture
for 48 hours after adding Olaparib (0.7M) and the r-APS.

| | Concentration | | |
|---|---|---|---|
| | 0 | 2 | 4 |
| Repeat | (mg/ml) | (mg/ml) | (mg/ml) |
| Repeat 1 | 0.598 | 0.252 | 0.224 |
| Repeat 2 | 0.584 | 0.244 | 0.216 |
| Repeat 3 | 0.602 | 0.255 | 0.210 |
| Mean value (MEAN) | 0.595 | 0.250 | 0.217 |
| Standard deviation (SD) | 0.009 | 0.006 | 0.006 |
| Cell viability (%) | 60.189 | 25.337 | 21.930 |

As shown in Table 10, the ovarian cancer cell line (OVCAR cells) has been cultured with Olaparib (0.7M) and the astragalus pharmaceutical composition comprising the r-APS and the mannose (r-APS+mannose) for 48 hours, the result shows that the astragalus pharmaceutical composition comprising the r-APS and the mannose (r-APS+mannose) in combination with chemotherapy drug can effectively inhibit the cell viability of ovarian cancer cell line (OVCAR cells), so the astragalus pharmaceutical composition comprising the r-APS and the mannose (r-APS+mannose) in combination with chemotherapy drug has a significant poisoning effect (as shown in FIG. 5E).

TABLE 10
Cell viability (%) of ovarian cancer cell line (OVCAR
cells) culture for 48 hours after adding Olaparib (0.7M)
and the astragalus pharmaceutical composition comprising
the r-APS and the mannose (r-APS + mannose)

| | Concentration | | |
|---|---|---|---|
| | 0 | 5 | 10 |
| Repeat | (mg/ml) | (mg/ml) | (mg/ml) |
| Repeat 1 | 0.598 | 0.220 | 0.183 |
| Repeat 2 | 0.584 | 0.205 | 0.185 |
| Repeat 3 | 0.632 | 0.227 | 0.175 |
| Mean value (MEAN) | 0.605 | 0.217 | 0.181 |
| Standard deviation (SD) | 0.025 | 0.011 | 0.005 |
| Cell viability (%) | 61.4 | 22.1 | 18.4 |

The experimental results show that the astragalus pharmaceutical composition comprising the r-APS and the mannose (r-APS+mannose) in combination with chemotherapy drug of the present invention can further reduce the viability of cancer cells of colorectal cancer by 43% compared with only using drug, so the therapeutic effect of chemotherapy drug can be enhanced effectively.

To sum up, the astragalus pharmaceutical composition comprising the r-APS and the mannose (r-APS+mannose) in combination with chemotherapy drug of the present invention can reduce the cell viability of cancer cells by at least 16%, so the ability of chemotherapy drug to reduce the cell viability of cancer cells can be enhanced effectively, and the proliferation of tumor cells can be inhibited effectively, the chemotherapeutic effect is stronger than only using r-APS, even on drug resistant cancers, the therapeutic effect of chemotherapy drug can be maintained or enhanced.

All examples provided herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority or inferiority of the invention. Although one or more embodiments of the present invention have been described in detail, it should be understood that the various changes, substitutions, and alterations could be made hereto without departing from the spirit and scope of the invention.

It is intended that the specification and examples be considered as examples only, with a true scope and spirit of the invention being indicated by the following claims.

1. A method for enhancing the treatment of a chemotherapy drug for a cancer in a subject for inhibiting the viability of a cancer cell, comprising administering to the subject an astragalus pharmaceutical composition, wherein the astragalus pharmaceutical composition comprises a refined astragalus polysaccharides (r-APS) and a mannose.

2. The method of claim 1, wherein the chemotherapy drug for the cancer is a targeted chemotherapy drug, wherein the targeted chemotherapy drug is a targeted chemotherapy drug for a corresponding cancer type.

3. The method of claim 1, wherein the cancer comprises colorectal cancer, ovarian cancer, or lung cancer.

4. The method of claim 2, wherein the targeted chemotherapy drug for the corresponding cancer type comprises Regorafenib, Olaparib or Tarceva.

5. The method of claim 1, wherein an effective dose ratio of the r-APS to the mannose is 1.5˜4.5:5.5˜8.5, and a concentration range of the astragalus pharmaceutical composition comprising the r-APS and the mannose is 0˜10 mg/mL.

6. The method of claim 1, wherein the form of the astragalus pharmaceutical composition can be tablet, pill, granule, powder, capsule, or liquid.

7. A method for enhancing the treatment of a chemotherapy drug for a cancer in a subject for treating a drug resistant cancer on inhibiting the viability of a cancer cell, comprising administering to the subject an astragalus pharmaceutical composition, wherein the astragalus pharmaceutical composition comprises a refined astragalus polysaccharides (r-APS) and a mannose.

8. The method of claim 7, wherein the chemotherapy drug for the cancer is a targeted chemotherapy drug, wherein the targeted chemotherapy drug is a targeted chemotherapy drug for a corresponding cancer type.

9. The method of claim 8, wherein the targeted chemotherapy drug for the corresponding cancer type comprises Regorafenib, Olaparib or Tarceva.

10. The method of claim 7, wherein the cancer is solid tumor cancer.

11. The method of claim 8, wherein the solid tumor cancer comprises colorectal cancer, ovarian cancer, or lung cancer.

12. The method of claim 7, wherein an effective dose ratio of the r-APS to the mannose is 1.5˜4.5:5.5˜8.5, and a concentration range of the r-APS and the mannose is 0˜10 mg/mL.

13. A method for enhancing the treatment of a chemotherapy drug for a cancer in a subject for inhibiting the proliferation of a tumor cell, comprising administering to the subject an astragalus pharmaceutical composition, wherein the astragalus pharmaceutical composition comprises a refined astragalus polysaccharides (r-APS) and a mannose.

14. The method of claim 13, wherein the tumor cell is a drug resistant tumor cell.

15. The method of claim 13, wherein the chemotherapy drug for the cancer is a targeted chemotherapy drug, wherein the targeted chemotherapy drug is a targeted chemotherapy drug for a corresponding cancer type.

16. The method of claim 15, wherein the targeted chemotherapy drug for the corresponding cancer type comprises Regorafenib, Olaparib or Tarceva.

17. The method of claim 13, wherein the tumor cell comprises colorectal cancer, ovarian cancer, or lung cancer.

18. The method of claim 13, wherein an effective dose ratio of the r-APS to the mannose is 1.5˜4.5:5.5˜8.5, and a concentration range of the astragalus pharmaceutical composition comprising the r-APS and the mannose is 0˜10 mg/mL.
